(19) United States
(12) Patent Application Publication
(10) Pub. No.: US 2008/0243008 A1
(43) Pub. Date: Oct. 2, 2008

(54) Blood Pressure Measuring Apparatus and Blood Pressure Measuring Method

(75) Inventors: Yoshiyuki Habu, Kanagawa-ken (JP); Kouji Hagi, Kanagawa-ken (JP); Hitoshi Ozawa, Kanagawa-ken (JP); Kimihisa Aihara, Kanagawa-ken (JP); Naoe Tatara, Kanagawa-ken (JP); Shinji Mino, Kanagawa-ken (JP); Hiroshi Koizumi, Kanagawa-ken (JP)

Correspondence Address: BUCHANAN, INGERSOLL & ROONEY PC, POST OFFICE BOX 1404, ALEXANDRIA, VA, 22313-1404, US

(73) Assignees: TERUMO KABUSHIKI KAISHA, Tokyo (JP); NIPPON TELEGRAPH AND TELEPHONE CORPORATION, Tokyo (JP)

(21) Appl. No.: 11/664,690

(22) PCT Filed: Oct. 3, 2005
(86) PCT No.: PCT/JP05/18293

§ 371 Date: Apr. 7, 2008

(30) Foreign Application Priority Data
Oct. 6, 2004 (JP) 2004-294307
Oct. 6, 2004 (JP) 2004-294308

(51) Int. Cl. A61B 5/02

(52) U.S. Cl. 600/494; 600/485; 600/493

(57) ABSTRACT

To provide a technique which makes it possible to obtain a proper pulse wave signal for high-accuracy blood pressure measurements, thereby saving the trouble of taking measurements repeatedly and reducing a physical burden imposed on the user by cuff pressure. A blood pressure measuring apparatus includes a cuff which is attached to and around an external ear; a first pulse wave detector and a second pulse wave detector which detect a pulse wave in a part squeezed by the cuff and which are affected differently from each other by a characteristic of body movements; a body movement detecting means which detects the characteristic of body movements; a pulse wave selecting means which selects a pulse wave detected by one of the first pulse wave detector and the second pulse wave detector based on the characteristic of body movements detected by the body movement detecting means; and a blood pressure value deriving means which derives a blood pressure value based on the pulse wave selected by the pulse wave selecting means.

### TECHNICAL FIELD

The present invention relates to a technique which makes it possible to derive blood pressures with high accuracy, particularly in blood pressure measurement of an external ear and its surroundings.

### BACKGROUND ART

Conventional blood pressure measuring apparatuses which use pulse waves are roughly classified into the photoplethysmography type, pressure plethysmography type, and Korotkoff type, according to their measurement principles. With the photoplethysmography type, light reflected by blood flowing through a part squeezed by a cuff is obtained as a pulse wave signal by a photosensor. With the pressure plethysmography type, the oscillation of blood vessel walls caused by blood flowing through a part squeezed by a cuff is obtained as a pulse wave signal by a pressure sensor. With the Korotkoff type, Korotkoff sounds produced due to squeezing by a cuff are obtained as a pulse wave signal by a microphone installed near the cuff. Blood pressure is measured as the variation of the obtained pulse wave signal with time.

However, under any of the above described measurement principles, measurement noise can occur as a direct or indirect result of body movements. Thus, a method has been proposed which takes measurements by using multiple measurement systems based on different measurement principles or by switching among them, and selects the most probable result based on human judgment, as described in Patent Document 1.

Also, when the pulse wave signal is weak or greatly saturated for some reason, making it impossible to take proper blood pressure measurements, an error signal is generated, prompting the user to change the mounting position of a pulse wave detection sensor before applying pressure and taking blood pressure measurements again or, as described in Patent Document 2, a signal level is adjusted by means of signal amplification or the like before applying pressure and taking blood pressure measurements again.

Patent Document 1: Japanese Patent No. 3240324

Patent Document 2: Japanese Publication of Examined Patent Application No. 6-18555

### DISCLOSURE OF INVENTION

Conventionally, however, if a proper pulse wave signal is not obtained, it is necessary to take measurements again, for example, after changing the cuff position. This is troublesome. This imposes a physical burden on the patient, who must go through multiple measurement operations, and be squeezed by the cuff multiple times.

The present invention has been made in view of the above problems and has as an object to provide a blood pressure measuring apparatus and blood pressure measuring method which make it possible to obtain a proper pulse wave signal for high-accuracy blood pressure measurements, thereby saving the trouble of taking measurements repeatedly and reducing the physical burden imposed on the user.

A blood pressure measuring apparatus comprises: a cuff which is attached to and around an external ear, a first pulse wave detector and a second pulse wave detector which detect a pulse wave in a part squeezed by the cuff and which are affected differently from each other by a characteristic of body movements, a body movement detecting means which detects the characteristic of body movements, pulse wave selecting means which selects a pulse wave detected by one of the first pulse wave detector and the second pulse wave detector based on the characteristic of body movements detected by the body movement detecting means, and blood pressure value deriving means which derives a blood pressure value based on the pulse wave selected by the pulse wave selecting means.

In the blood pressure measuring apparatus, the body movement detecting means comprises level detecting means which detects the magnitude of the body movements, and the pulse wave selecting means selects a pulse wave to derive blood pressure based on the magnitude of body movements detected by the level detecting means.

Also, the body movement detecting means further comprises a period detecting means which detects a period of the body movements; and the pulse wave selecting means selects a pulse wave to derive blood pressure based on the magnitude of the body movements detected by the level detecting means and the period of the body movements detected by the period detecting means.

A blood pressure measuring apparatus comprises: a first cuff which is attached to and around an external ear, a first pulse wave detector and a second pulse wave detector which detect a pulse wave in a part squeezed by the first cuff and which are affected differently from each other by a characteristic of body movements, a body movement detecting means which detects the characteristic of body movements, first pulse wave selecting means which selects a pulse wave detected by one of the first pulse wave detector and the second pulse wave detector based on the characteristic of body moments detected by the body movement detecting means, a first blood pressure value deriving means which derives a blood pressure value based on the pulse wave selected by the first pulse wave selecting means, a second cuff mounted in a different location from the first cuff, a blood pressure determining means which determines blood pressure by detecting a pulse wave in a part squeezed by the second cuff, and pressurization control means which synchronizes pressurization of the first cuff and the second cuff.

In the blood pressure measuring apparatus, the blood pressure determining means comprises: a third pulse wave detector and a fourth pulse wave detector which detect a pulse wave in the part squeezed by the second cuff and which are affected differently from each other by a characteristic of body movements, second pulse wave selecting means which selects a pulse wave detected by one of the third pulse wave detector and the fourth pulse wave detector based on the characteristic of body movements detected by the body movement detecting means, and a second blood pressure value deriving means which derives a blood pressure value based on the pulse wave selected by the second pulse wave selecting means.

A blood pressure measuring apparatus comprises: a cuff which is attached to and around an external ear, a pulse wave detector which detects a pulse wave in a part squeezed by the cuff, level control means which controls a signal level of the pulse wave, and a blood pressure derivation control means which makes adjustments using the level control means so as to correct any deviation of the signal level of the pulse wave detected by the pulse wave detector during compression of the cuff from a predetermined range, and derives a blood pressure value based on the pulse wave detected by the pulse wave detector during decompression of the cuff.

In the blood pressure measuring apparatus, if the signal level of the pulse wave detected by the pulse wave detector during compression of the cuff falls within the predetermined range, the blood pressure derivation control means derives a blood pressure value based on the pulse wave and finishes a measurement operation.

Also, the pulse wave detected by the pulse wave detector is a photoelectric volume pulse wave obtained via light absorption and reflection by blood in blood vessels.

Furthermore, the level control means comprises at least one of a light quantity adjusting means which adjusts the quantity of output light from a light-emitting element which emits light to the blood in the blood vessels and a gain control means which controls a signal level from a light-receiving element which detects absorption and reflection of the light from the light-emitting element by the blood in the blood vessels.

A blood pressure measuring apparatus comprises: a cuff which is attached to and around an external ear, a pulse wave detector which detects a pulse wave in a part squeezed by the cuff, a level control means which controls a signal level of the pulse wave, and a blood pressure derivation control means which makes adjustments using the level control means so as to correct any deviation of the signal level of the pulse wave detected by the pulse wave detector before compression or at an early stage of compression of the cuff from a predetermined range, and derives a blood pressure value based on the pulse wave detected by the pulse wave detector during subsequent compression of the cuff.

In the blood pressure measuring apparatus, the pulse wave detected by the pulse wave detector is a photoelectric volume pulse wave obtained via light absorption and reflection by blood in blood vessels.

Furthermore, the level control means comprises at least one of a light quantity adjusting means which adjusts the quantity of output light from a light-emitting element which emits light to the blood in the blood vessels and a gain control means which controls a signal level from a light-receiving element which detects absorption and reflection of the light from the light-emitting element by the blood in the blood vessels.

A blood pressure measuring apparatus comprises a first cuff which is attached to and around an external ear, a pulse wave detector which detects a pulse wave in a part squeezed by the first cuff, level control means which controls a signal level of the pulse wave, a blood pressure derivation control means which makes adjustments using the level control means so as to correct any deviation of the signal level of the pulse wave detected by the pulse wave detector during compression of the first cuff from a predetermined range, and derives a blood pressure value based on the pulse wave detected by the pulse wave detector during decompression of the first cuff, a second cuff mounted in a different location from the first cuff, a blood pressure determining means which determines blood pressure by detecting a pulse wave in a part squeezed by the second cuff, and a pressurization control means which synchronizes pressurization of the first cuff and the second cuff.

A blood pressure measuring apparatus comprises a first cuff which is attached to and around an external ear, a pulse wave detector which detects a pulse wave in a part squeezed by the first cuff, a level control means which controls a signal level of the pulse wave a blood pressure derivation control means which makes adjustments using the level control means so as to correct any deviation of the signal level of the pulse wave detected by the pulse wave detector before compression or at an early stage of compression of the first cuff from a predetermined range and derives a blood pressure value based on the pulse wave detected by the pulse wave detector during subsequent compression of the first cuff, a second cuff mounted in a different location from the first cuff, and a blood pressure determining means which determines blood pressure by detecting a pulse wave in a part squeezed by the second cuff, and a pressurization control means which synchronizes pressurization of the first cuff and the second cuff.

A blood pressure measuring method comprises: a pulse wave detecting step of detecting a first pulse wave and a second pulse wave in a part squeezed by a cuff attached to and around an external ear, the first pulse wave and the second pulse wave being affected differently from each other by a characteristic of body movements, a body movement detecting step of detecting the characteristic of body movements, a pulse wave selecting step of selecting one of the first pulse wave and the second pulse wave based on the characteristic of body movements detected by the body movement detecting step, and a blood pressure value deriving step of deriving a blood pressure value based on the pulse wave selected by the pulse wave selecting step.

In the blood pressure measuring method, the body movement detecting step comprises a level detecting step of detecting the magnitude of the body movements, and the pulse wave selecting step selects a pulse wave to derive blood pressure based on the magnitude of the body movements detected by the level detecting step.

Also, the body movement detecting step further comprises a period detecting step of detecting a period of the body movements, and the pulse wave selecting step selects a pulse wave to derive blood pressure based on the magnitude of the body movements detected by the level detecting step and the period of the body movements detected by the period detecting step.

A blood pressure measuring method comprises: a compression-time pulse wave detecting step of detecting a pulse wave in a part squeezed by a cuff attached to and around an external ear during compression of the cuff, a level control step of controlling a signal level of the pulse wave so as to correct any deviation of the signal level of the pulse wave detected by the compression-time pulse wave detecting step from a predetermined range, a decompression-time pulse wave detecting step of detecting a pulse wave in a part squeezed by the cuff during decompression of the cuff, and a blood pressure value deriving step of deriving a blood pressure value based on a pulse wave whose signal level, as detected by the compression-time pulse wave detecting step or the decompression-time pulse wave detecting step, falls within a predetermined range.

In the blood pressure measuring method, if the signal level of the pulse wave detected by the compression-time pulse wave detecting step falls within a predetermined range, the blood pressure value deriving step derives a blood pressure value based on the pulse wave detected by the compression-time pulse wave detecting step without regard to the level control step and the decompression-time pulse wave detecting step.

Also, the pulse waves detected by the compression-time pulse wave detecting step and the decompression-time pulse wave detecting step are photoelectric volume pulse waves obtained via light absorption and reflection by blood in blood vessels.

Furthermore, the level control step comprises at least one of a light quantity adjusting step of adjusting the quantity of output light from a light-emitting element which emits light to the blood in the blood vessels and a gain control step of controlling a signal level from a light-receiving element which detects absorption and reflection of the light from the light-emitting element by the blood in the blood vessels.

A blood pressure measuring method comprises: an initial pulse wave detecting step of detecting a pulse wave in a part squeezed by a cuff attached to and around an external ear before compression or at an early stage of compression of the cuff, a level control step of controlling a signal level of the pulse wave so as to correct any deviation of the signal level of the pulse wave detected by the initial pulse wave detecting step from a predetermined range, a pulse wave detecting step of detecting a pulse wave in the part squeezed by the cuff during subsequent compression of the cuff, and a blood pressure value deriving step of deriving a blood pressure value based on the pulse wave detected by the pulse wave detecting step.

In the blood pressure measuring method, the pulse wave detected by the pulse wave detecting step is a photoelectric volume pulse wave obtained via light absorption and reflection by blood in the blood vessels.

Also, the level control step comprises at least one of a light quantity adjusting step of adjusting the quantity of output light from a light-emitting element which emits light to the blood in the blood vessels and a gain control step of controlling a signal level from a light-receiving element which detects absorption and reflection of the light from the light-emitting element by the blood in the blood vessels.

### EFFECT OF THE INVENTION

The present invention provides a technique which makes it possible to easily obtain a proper pulse wave signal for blood pressure measurements.

Other features and advantages of the present invention will be apparent from the following description taken in conjunction with the accompanying drawings, in which like reference numerals denote the same or similar parts throughout the figures thereof.

### BRIEF DESCRIPTION OF DRAWINGS

The accompanying drawings, which are incorporated herein and form part of the specification, illustrate embodiments of the present invention and, together with the description, serve to explain the principles of the present invention.

FIG. 1 is an internal block diagram of a blood pressure measuring apparatus according to a first embodiment;

FIG. 2 is a diagram showing structure and operation in a cuff;

FIG. 3 is an external perspective view of the blood pressure measuring apparatus according to the first embodiment;

FIG. 4A is an operation flowchart of the blood pressure measuring apparatus according to the first embodiment;

FIG. 4B is an operation flowchart of the blood pressure measuring apparatus according to the first embodiment;

FIG. 5 is a diagram showing exemplary choices of pulse waves based on a characteristic of body movements for a blood pressure measuring apparatus according to a second embodiment;

FIG. 6 is an internal block diagram of a blood pressure measuring apparatus according to a third embodiment;

FIG. 7 is a diagram showing a cuff attached to and around a tragus;

FIG. 8 is an internal block diagram of a blood pressure measuring apparatus according to a fourth embodiment;

FIG. 9A is an operation flowchart of the blood pressure measuring apparatus according to the fourth embodiment;

FIG. 9B is an operation flowchart of the blood pressure measuring apparatus according to the fourth embodiment;

FIG. 10 is an operation flowchart of signal level adjustment in the blood pressure measuring apparatus according to the fourth embodiment;

FIG. 11 is a diagram showing cuff pressure and a pulse wave signal during blood pressure measurements in an exemplary fashion;

FIG. 12 is an exemplary circuit diagram related to signal level adjustment;

FIG. 13A is an operation flowchart of a blood pressure measuring apparatus according to a fifth embodiment;

FIG. 13B is an operation flowchart of the blood pressure measuring apparatus according to the fifth embodiment; and

FIG. 14 is an internal block diagram of a blood pressure measuring apparatus according to a sixth embodiment.

### BEST MODE FOR CARRYING OUT THE INVENTION

Preferred embodiments of the present invention will be described in detail below in an exemplary fashion with reference to the drawings. However, the components described in the embodiments are only exemplary and are not intended to limit the scope of the present invention.

### First Embodiment

A first embodiment of a blood pressure measuring apparatus according to the present invention will be described by citing a sphygmomanometer which uses a tragus and its surroundings as a measurement site.

<Equipment Configuration>

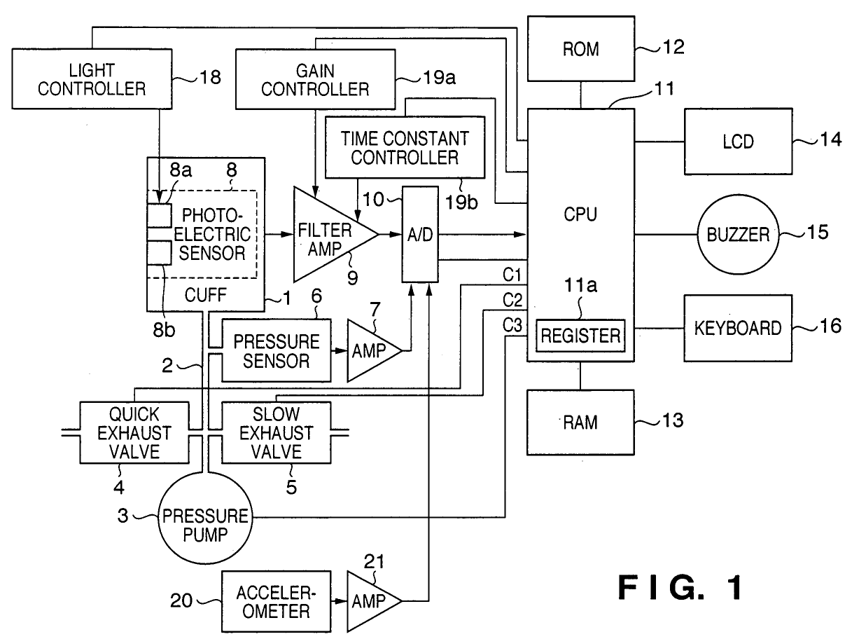

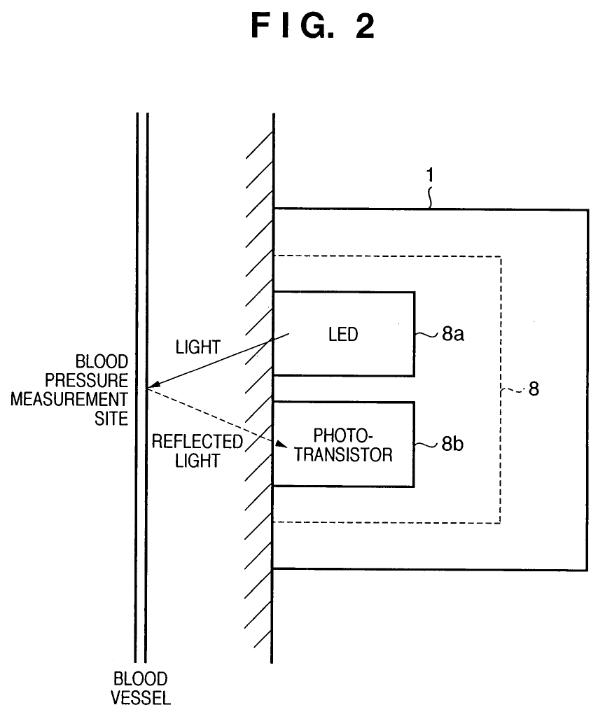

FIG. 1 is an internal block diagram of a blood pressure measuring apparatus according to the first embodiment. FIG. 2 is a diagram showing structure and operation in a cuff.

Reference numeral 1 denotes a cuff which is secured to a blood pressure measurement site so that it can squeeze blood vessels. Reference numeral 2 denotes a rubber tube which constitutes an air flow path into the cuff 1. Reference numeral 3 denotes a pressure pump which delivers compressed air into the cuff 1. Reference numeral 4 denotes a quick exhaust valve which reduces pressure in the cuff 1 quickly. Reference numeral 5 denotes a slow exhaust valve which reduces pressure in the cuff 1 at a constant rate (2 to 3 mmHg/sec). Reference numeral 6 denotes a pressure sensor which varies an electrical parameter according to the pressure in the cuff 1. Reference numeral 7 denotes a pressure pulse wave detection amplifier (AMP) which detects the electrical parameter from the pressure sensor 6, converts it into an electrical signal, amplifies it, and outputs an analog cuff pressure signal P.

Reference numeral 8 denotes a pulse wave sensor installed in the cuff 1. The pulse wave sensor 8 includes an LED 8a which illuminates pulsating vascular blood flow with light and a phototransistor 8b which detects light reflected by the vascular blood flow. Reference numeral 9 denotes a photoelectric pulse wave detection amplifier (AMP) which amplifies an output signal from the phototransistor 8b and outputs an analog pulse wave signal M. The LED 8a is connected with a light controller 18 which automatically varies the light quantity. On the other hand, the photoelectric pulse wave detection amplifier 9 is connected with a gain controller 19a which varies gain and a time constant controller 19b which varies a time constant of the amplifier 9. Also, the blood pressure measuring apparatus has an accelerometer 20 and body movement detection amplifier (AMP) 21 to detect body movements. They output an acceleration signal A. Reference numeral 10 denotes an A/D converter (A/D) which converts analog signals M, P, and A (not shown) into digital data D (not shown).

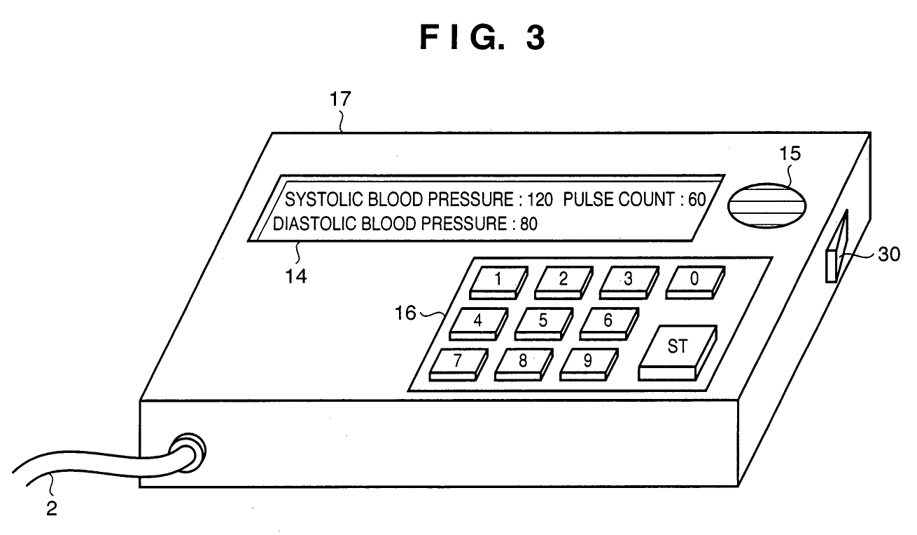

Reference numeral 11 denotes a controller (CPU) which performs main control of this blood pressure measuring apparatus. The CPU 11 has an adjustment pressure register 11a which stores adjustment pressure. Details of control will be described later. Reference numeral 12 denotes a ROM which stores a control program (such as that shown in FIG. 3) executed by the CPU 11. Reference numeral 13 denotes a RAM which has a data memory, image memory, and the like. Reference numeral 14 denotes a liquid crystal display (LCD) which displays the contents of the image memory. Reference numeral 16 denotes a keyboard which allows the user to enter a measurement start command, set an adjustment pressure value, and so on. Reference numeral 15 denotes a buzzer which informs the user that the apparatus has sensed the activation of a key on a keyboard 16, that measurements have been done, and so on. Incidentally, although the adjustment pressure register 11a is installed in the CPU 11 in this example, an adjustment pressure storage unit may be installed in the RAM 13.

FIG. 3 is an external perspective view of the blood pressure measuring apparatus according to the first embodiment. Reference numeral 17 denotes a main body of the sphygmomanometer, which contains components other than the cuff 1 and pulse wave sensor 8 in FIG. 1. In FIG. 3, the rubber tube (air tube) 2 includes a signal line for communication with the pulse wave sensor 8. It is connected to the cuff 1 and pulse wave sensor 8 (both not shown). The LCD display panel 14 is a dot-matrix display panel, and can display various information (e.g., characters, graphics, signal waveforms, and the like). Reference numeral 30 denotes a power switch. The keyboard 16 has a measurement start switch (ST) as well as a numeric keypad to enter a pressure value of the cuff and the like.

<Attaching the Cuff to a Measurement Site>

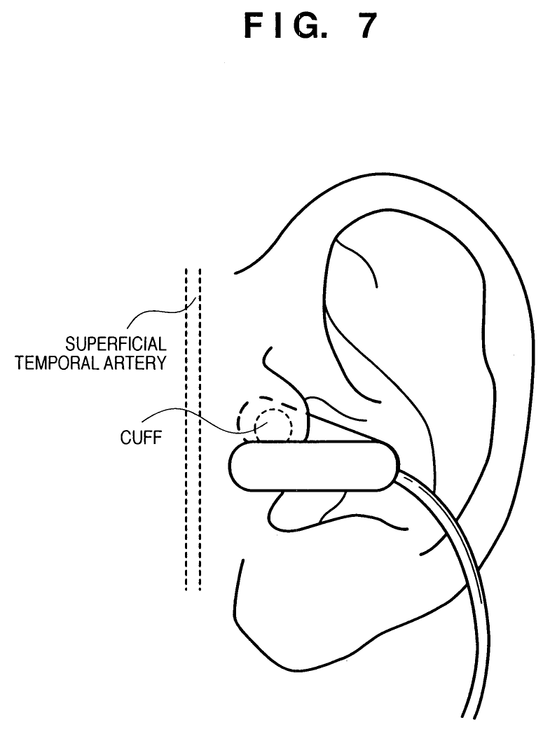

FIG. 7 is a diagram showing a cuff attached to and around a tragus. Since a tragus and its surroundings are used as a measurement site, a measuring unit including the cuff is configured to squeeze the tragus by pinching it from both sides. Incidentally, since movements of the measurement site have the most impact on blood pressure values, the accelerometer 20 is preferably mounted near the mounting location of the measuring unit or mounted integrally with the measuring unit.

<Equipment Operation>

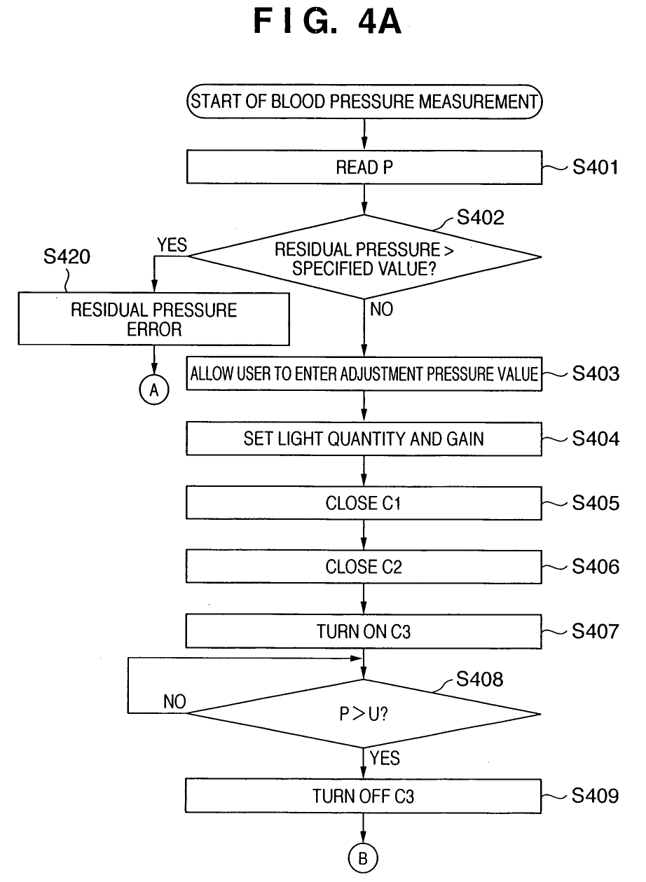

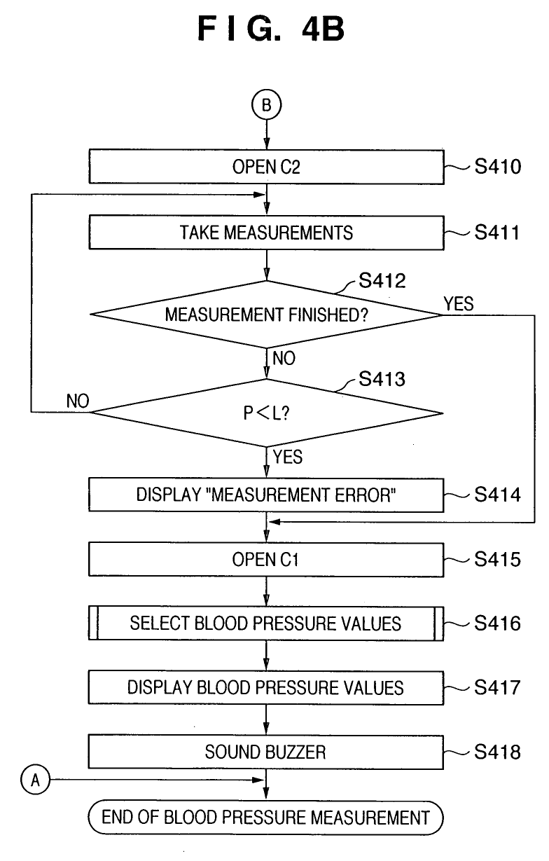

FIGS. 4A and 4B are operation flowcharts of the blood pressure measuring apparatus according to the first embodiment.

When the apparatus is powered on, it initializes itself by performing a self-diagnosis process (not shown). Subsequently, when the measurement start switch ST is pressed, the apparatus starts processing.

In Step S401, the apparatus reads the cuff pressure P. In Step S402, the apparatus compares residual pressure of the cuff 1 with a specified value. If the residual pressure exceeds the specified value, the apparatus displays “residual pressure error” on the LCD 14 in Step S420. If the residual pressure is not higher than the specified value, the apparatus allows the user in Step S403 to set a pressurization value (e.g., a value between 120 and 210 mmHg, which is higher than a systolic blood pressure) using the keyboard 16. In Step S404, the apparatus sets the light quantity and gain to predetermined values.

When the light quantity and gain have been set, the apparatus closes the quick exhaust valve 4 and slow exhaust valve 5 in Steps S405 and S406, respectively. In Step S407, the apparatus starts operating the pressure pump 3, and thereby starts pressurization (compression).

In Step S408, the apparatus determines whether or not the cuff pressure is higher than the pressurization value U set in Step S403. If P>U is not met, the apparatus continues pressurization. If P>U, the apparatus stops the pressure pump 3 in Step S409.

In Step S410, the apparatus opens the slow exhaust valve 5. This marks the start of a measurement step during depressurization (decompression). The cuff pressure starts to fall at a constant rate (e.g., 2 to 3 mmHg/sec). At the same time, a body movement detecting means (accelerometer) starts detecting acceleration and a first blood pressure determining means (photoplethysmography type) and second blood pressure determining means (pressure plethysmography type) start detecting pulse waves. Incidentally, the first blood pressure determining means illuminates blood vessels with light from the light-emitting element 8a, receives light reflected by the blood vessels using the light-receiving element 8b, and detects the light quantity (quantity of reflection which varies depending on the blood flow rate in the blood vessels) as a photoelectric pulse wave. At the same time, the second blood pressure determining means detects air pressure in the cuff, i.e., oscillation amplitude which varies with the amount of squeeze (air pressure which oscillates with oscillation of blood vessel walls corresponding to pulsation), as a pressure pulse wave using the pressure sensor. Meanwhile, in Step S411, various functional blocks perform data processing, and the apparatus measures systolic and diastolic blood pressures by the application of predetermined algorithms to a photoelectric pulse wave signal and pressure pulse wave signal. In Step S412, the apparatus determines whether or not a diastolic blood pressure value during depressurization has been detected. If both the diastolic blood pressure value measured from photoelectric pulse wave data and the diastolic blood pressure value measured from pressure pulse wave data have not been detected, the apparatus continues measurement. In Step S413, the apparatus determines whether or not the cuff pressure is lower than a predetermined value L (e.g., 40 mmHg). If P<L is not met, the cuff pressure is within a normal measuring range and thus the flow returns to Step S411. On the other hand, if P<L, the cuff pressure is already lower than the normal measuring range. Thus, if normal data is not obtained from either the photoelectric pulse wave signal or pressure pulse wave signal (e.g., if a determined systolic blood pressure is not higher than 40 mmHg), the apparatus displays “measurement error” on the LCD 14 in Step S414. In so doing, the apparatus additionally displays detailed information such as “signal failure during depressurization” if necessary. In Step S415, the apparatus opens the quick exhaust valve 4.

In Step S416, the apparatus selects either the systolic and diastolic blood pressure values determined from the photoelectric pulse wave or the systolic and diastolic blood pressure values determined from the pressure pulse wave depending on whether a value obtained by the accelerometer exceeds a predetermined value C. It is desirable to select the systolic and diastolic blood pressure values determined from the photoelectric pulse wave by determining that accurate blood pressure cannot be obtained from the pressure pulse wave due to body movements during measurement if the predetermined value C is exceeded, or select the systolic and diastolic blood pressure values determined from the pressure pulse wave if the predetermined value C is not exceeded. Incidentally, although the blood pressure values to be displayed are selected after deriving blood pressure values from each of the photoelectric pulse wave and pressure pulse wave, either the photoelectric pulse wave data or pressure pulse wave data may be selected before deriving blood pressure values.

In Step S417, the apparatus displays the selected systolic and diastolic blood pressure values on the LCD 14. In Step S418, the apparatus sounds a buzzer to inform the user of the end of measurements.

As described above, the sphygmomanometer according to this embodiment can objectively select proper blood pressure to be displayed out of blood pressure measurement results from the first blood pressure determining means of a photoplethysmography type and blood pressure measurement results from the second blood pressure determining means of a pressure plethysmography type based on the signal intensity from the accelerometer which is a body movement detecting means and using information as to whether a threshold corresponding to a predetermined acceleration has been exceeded as a judgment criterion. Incidentally, this embodiment is especially advantageous in measurements of a tragus and its surroundings, in which the effect of head movements cannot be ignored. Consequently, this embodiment can be applied easily to continuous measurement of blood pressure.

### Second Embodiment

A second embodiment further has a function to calculate, using the CPU 11, periodic components of body movements from data produced by the accelerometer which is a body movement period calculating means. Thus, this embodiment makes it possible to effectively select blood pressure values for display output out of the blood pressure values determined from the photoelectric pulse wave and the blood pressure values determined from the pressure pulse wave in Step S416 for the following reasons.

When photoplethysmographic and pressure plethysmographic blood pressure measuring methods are compared based on measurement principles, the pressure plethysmographic method, which uses air for detection, is impervious to disturbing oscillation attributable to short-period (rapid) body movements because the disturbing oscillation is attenuated by air while the photoplethysmographic method is susceptible to short-period body movements. Thus, the pressure plethysmographic method is more desirable for blood pressure measurements in the presence of short-period (rapid) body movements of a lower magnitude than a predetermined value.

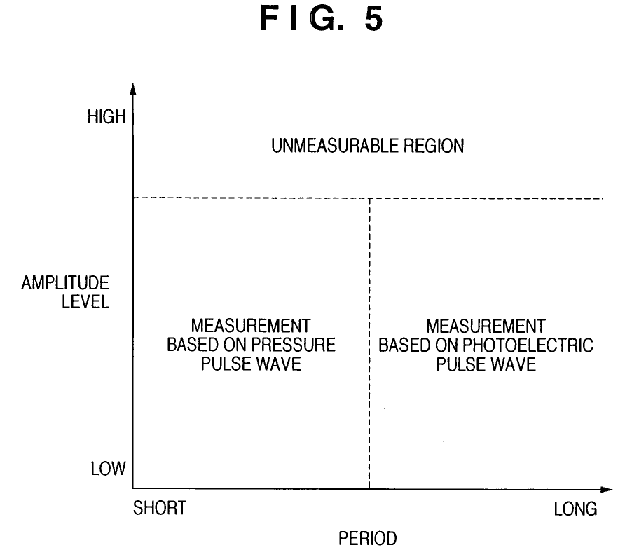

FIG. 5 is a diagram showing exemplary choices of pulse waves based on a characteristic of body movements for a blood pressure measuring apparatus according to the second embodiment. Incidentally, although a photoelectric pulse wave is selected at a low noise level in this example, a pressure pulse wave may be detected more stably depending on the measurement site. In that case, a pressure pulse wave may be selected at a low noise level.

### Third Embodiment

In a third embodiment, description will be given of a blood pressure measuring apparatus which can measure multiple sites at a time.

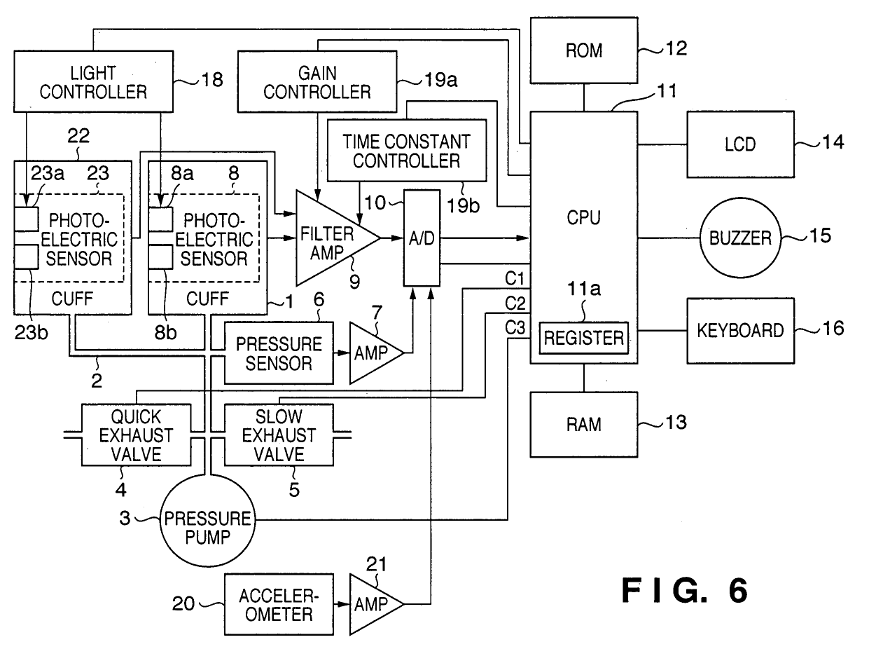

FIG. 6 is an internal block diagram of the blood pressure measuring apparatus according to the third embodiment. Each cuff which pinches a tragus and/or its surroundings is equipped with a light-emitting unit (see FIG. 6: LED 8a or 23a) and light-receiving unit (see FIG. 6: phototransistor 8b or 23b). The two cuffs are configured to be pressurized by a single pressure pump 3 to measure blood pressure at multiple sites on and/or around a tragus, i.e., the front and back sides of the tragus, simultaneously. Incidentally, sensors based on different measurement principles (the pressure plethysmographic method and the like) may be used for blood pressure measurements. The rest of the configuration and operation is the same as the first and second embodiments, and thus description thereof will be omitted.

It is known that blood vessels (arterioles) in and/or around the tragi are located in close vicinity to blood vessels in the brain, and it is considered that changes in blood pressure resulting from intracerebral causes can be measured. On the other hand, around the tragi, there are not only blood vessels (arterioles) in the ear cartilage (mainly tragi), but also arteries (superficial temporal artery) directly connected to the heart. This offers the advantage of being able to measure blood pressures carrying different pieces of information (blood pressure attributable to the brain and blood pressure attributable to the heart) simultaneously around a tragus using a small apparatus. The blood pressure measuring apparatus according to this embodiment makes it possible to objectively select the most probable result out of blood pressure measurement results produced by multiple methods, based on a characteristic of body movements during a period of blood pressure measurement, and thereby take high-accuracy blood pressure measurements around a tragus.

### Fourth Embodiment

A fourth embodiment of a blood pressure measuring apparatus according to the present invention will be described by citing a photoelectric sphygmomanometer which uses an appropriate location on and around an external ear as a measurement site.

<Equipment Configuration>

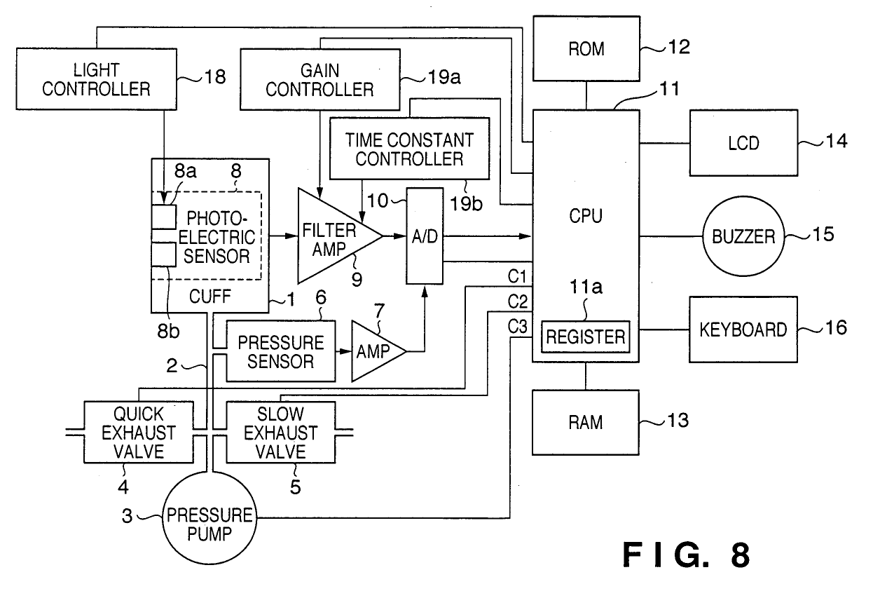

FIG. 8 is an internal block diagram of a blood pressure measuring apparatus according to the fourth embodiment. Reference numeral 1 denotes a cuff which is secured to a blood pressure measurement site around an external ear, and preferably to a tragus, so that it can squeeze blood vessels (arterioles) in and around the external ear. Reference numeral 2 denotes a rubber tube (air tube) which constitutes an air flow path into the cuff 1. Reference numeral 3 denotes a pressure pump which delivers compressed air into the cuff 1. Reference numeral 4 denotes a quick exhaust valve which reduces pressure in the cuff 1 quickly. Reference numeral 5 denotes a slow exhaust valve which reduces pressure in the cuff 1 at a constant rate (e.g., 2 to 3 mmHg/sec). Reference numeral 6 denotes a pressure sensor which varies an electrical parameter according to the pressure in the cuff 1. Reference numeral 7 denotes a pressure detection amplifier (AMP) which detects the electrical parameter from the pressure sensor 6, converts it into an electrical signal, amplifies it, and outputs an analog cuff pressure signal P.

Reference numeral 8 denotes a pulse wave sensor installed in the cuff 1. The pulse wave sensor 8 includes an LED 8a which illuminates pulsating vascular blood flow with light and a phototransistor 8b which detects light reflected by the vascular blood flow (FIG. 2). Reference numeral 9 denotes a pulse wave detection amplifier (AMP) which amplifies an output signal from the phototransistor 8b and outputs an analog pulse wave signal M. The LED 8a is connected with a light controller 18 which automatically varies the light quantity. On the other hand, the pulse wave detection amplifier 9 is connected with a gain controller 19a which varies gain as well as with a time constant controller 19b which varies a time constant of filter amplifiers 91 and 92 (described later) composing the pulse wave detection filter amplifier 9. Reference numeral 10 denotes an A/D converter (A/D) which converts analog signals M and P (not shown) into digital data D (not shown).

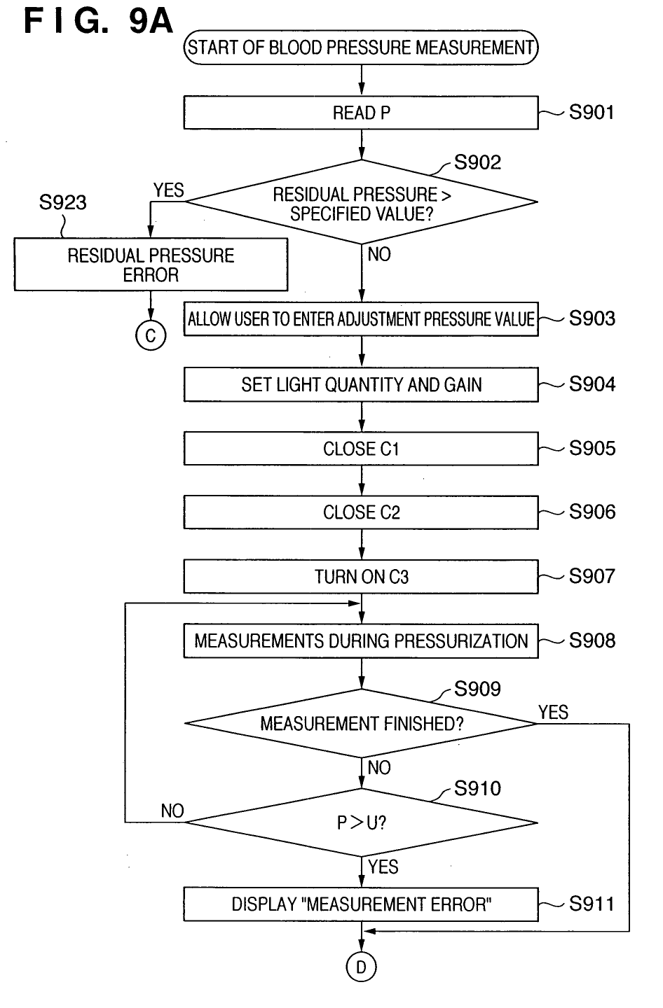

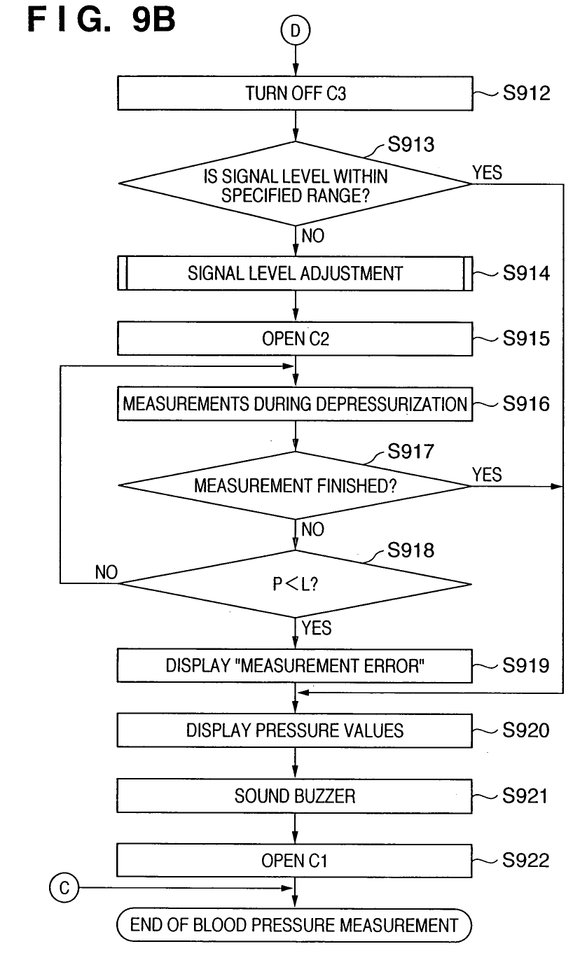

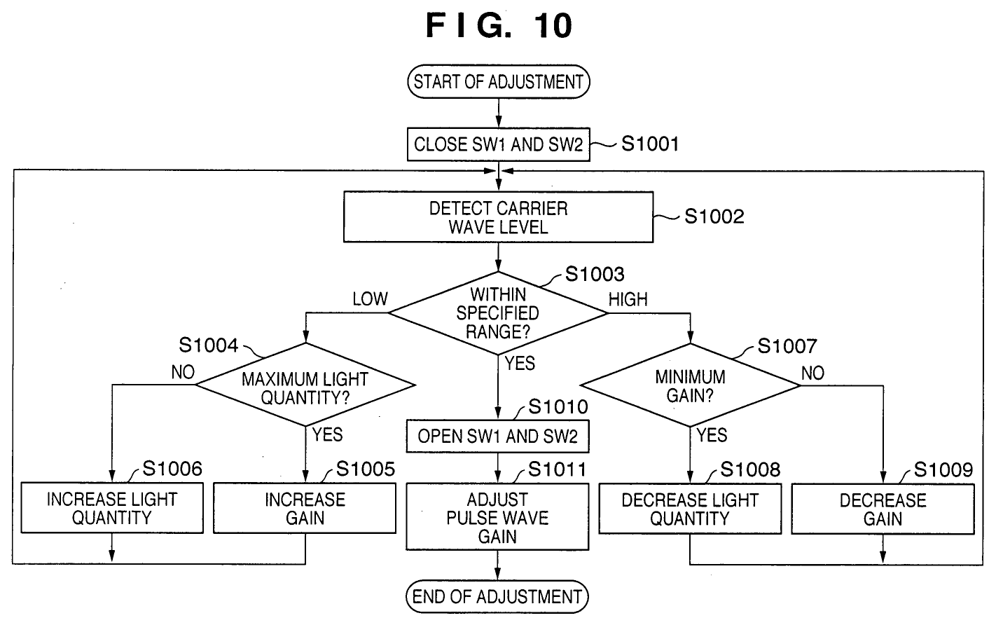

Reference numeral 11 denotes a controller (CPU) which performs main control of this photoelectric sphygmomanometer. The CPU 11 has an adjustment pressure register 11a which stores adjustment pressure. Details of this control will be described later. Reference numeral 12 denotes a ROM which stores a program for control (such as shown in FIGS. 9A, 9B, and 10) executed by the CPU 11. Reference numeral 13 denotes a RAM which has a data memory, image memory, and the like. Reference numeral 14 denotes a liquid crystal display (LCD) which displays the contents of the image memory. Reference numeral 16 denotes a keyboard which allows the user to enter a measurement start command, set an adjustment pressure value, and so on. Reference numeral 15 denotes a buzzer which informs the user that the apparatus has sensed the activation of a key on a keyboard 16, that measurements have been done, and so on. Incidentally, although the adjustment pressure register 11a is installed in the CPU 11 in this example, an adjustment pressure storage unit may be installed in the RAM 13.

### <Attaching the Cuff to a Measurement Site>

Since a tragus and its surroundings are used as a measurement site, a measuring unit including the cuff is configured to squeeze the tragus by pinching it from both sides as shown in FIG. 7.

<Equipment Operation>

FIGS. 9A and 9B are operation flowcharts of the blood pressure measuring apparatus according to the fourth embodiment. When the apparatus is turned on by the power switch 30, it initializes itself by performing a self-diagnosis process (not shown). Subsequently, when the measurement start switch ST is pressed, the apparatus starts processing.

In Step S901, the apparatus reads the cuff pressure P. In Step S902, the apparatus compares residual pressure of the cuff 1 with a specified value. If the residual pressure exceeds the specified value, the apparatus displays “residual pressure error” on the LCD 14 in Step S923. If the residual pressure is not higher than the specified value, the apparatus allows the user in Step S903 to set an upper limit of pressurization (e.g., a value between 120 and 280 mmHg, which is higher than a systolic blood pressure) using the keyboard 16. In Step S904, the apparatus sets the light quantity and gain to predetermined values.

When the light quantity and gain have been set, the apparatus closes the quick exhaust valve 4 and slow exhaust valve 5 in Steps S905 and S906, respectively. In Step S907, the apparatus starts operating the pressure pump 3, and thereby starts pressurization (compression). This marks the start of a measurement stroke during pressurization. The cuff pressure starts to increase at a constant rate (e.g., 2 to 3 mmHg/sec). Meanwhile, in Step S908, various functional blocks perform data processing, and the apparatus measures systolic and diastolic blood pressures. Once the systolic blood pressure has been measured (Step S909), the apparatus stops the pressure pump 3 in Step S912. In Step S910, the apparatus determines whether or not the cuff pressure is higher than the pressurization value U set in Step S903. If P>U is not met, the cuff pressure is still within a normal measuring range and thus the apparatus continues measurement. On the other hand, if P>U, the cuff pressure is already higher than the set value. Thus, the apparatus displays “measurement error” on the LCD 14 in Step S911. The apparatus additionally displays detailed information such as “signal failure during pressurization” if necessary. In Step S913, the apparatus determines whether or not the measured signal level is within a specified range. And, if the measured signal level is not within a specified range, advance to Step S914. In Step S914, the apparatus adjusts the light quantity and gain based on the signal level of a pulse wave signal obtained during pressurization.

When the adjustments of the light quantity and gain are finished, the apparatus opens the slow exhaust valve 5 in Step S915. This marks the start of a measurement stroke during depressurization. The cuff pressure starts to fall at a constant rate (e.g., 2 to 3 mmHg/sec). Meanwhile, in Step S916, various functional blocks perform data processing, and the apparatus measures systolic and diastolic blood pressures. In Step S917, the apparatus determines whether or not a diastolic blood pressure value during depressurization has been detected. If a diastolic blood pressure value has not been detected, the apparatus continues measurement. In Step S918, the apparatus determines whether or not the cuff pressure is lower than a predetermined value L (e.g., 40 mmHg). If P<L is not met, the cuff pressure is within a normal measuring range and thus the flow returns to Step S916. On the other hand, if P<L, the cuff pressure is already lower than the normal measuring range. Thus, the apparatus displays “measurement error” on the LCD 14 in Step S919. The apparatus additionally displays detailed information such as “signal failure during depressurization” if necessary.

If it is found in Step S917 that measurements have been finished, meaning that the measurement stroke has been finished within a normal measuring range, the apparatus displays the measured systolic and diastolic blood pressures on the LCD 14 in Step S920 and sends a tone signal to the buzzer 15 in Step S921. Preferably, different tone signals are sent for a normal end and an abnormal end. In Step S922, the apparatus discharges remaining air from the cuff 1 and waits for the next measurements to start.

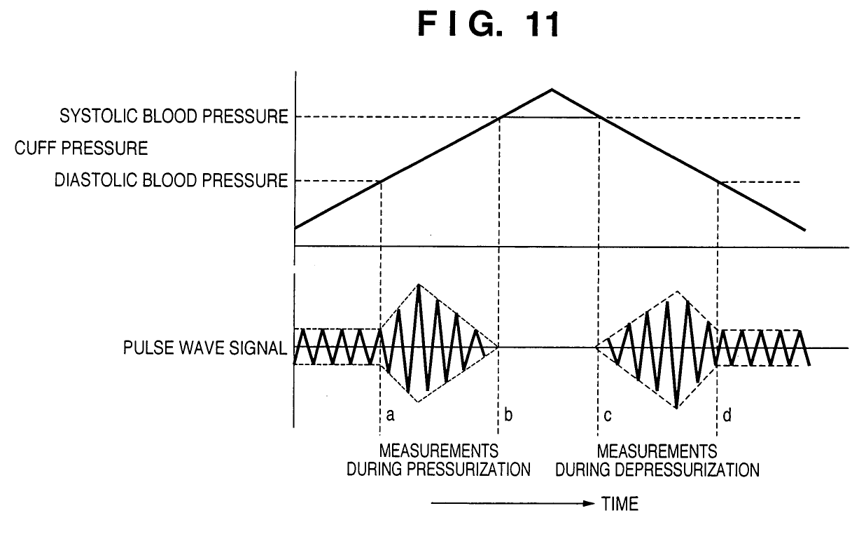

FIG. 11 is a diagram showing cuff pressure and a pulse wave signal during blood pressure measurements in an exemplary fashion. It shows the cuff pressure and pulse wave signal detected by a velocity (variation detection) sensor during a period from the start of measurements during pressurization (Step S908) to the end of measurements during depressurization (Step S916).

Blood pressure values are derived approximately as follows based on changes in the pulse wave signal shown in FIG. 11. Specifically, in the measurements during pressurization, the cuff pressure at a point (a) at which the magnitude of the pulse wave signal starts to change is designated as the systolic blood pressure and the cuff pressure at a point (b) at which the pulse wave signal extinguishes is designated as the diastolic blood pressure. Contrary to the measurements during pressurization, in the measurements during depressurization, the cuff pressure at a point (c) at which the pulse wave signal appears is designated as the systolic blood pressure and the cuff pressure at a point (d) at which the magnitude of the pulse wave signal stops changing is designated as the diastolic blood pressure.

<Details of Adjusting the Light Quantity and Gain of the Apparatus>

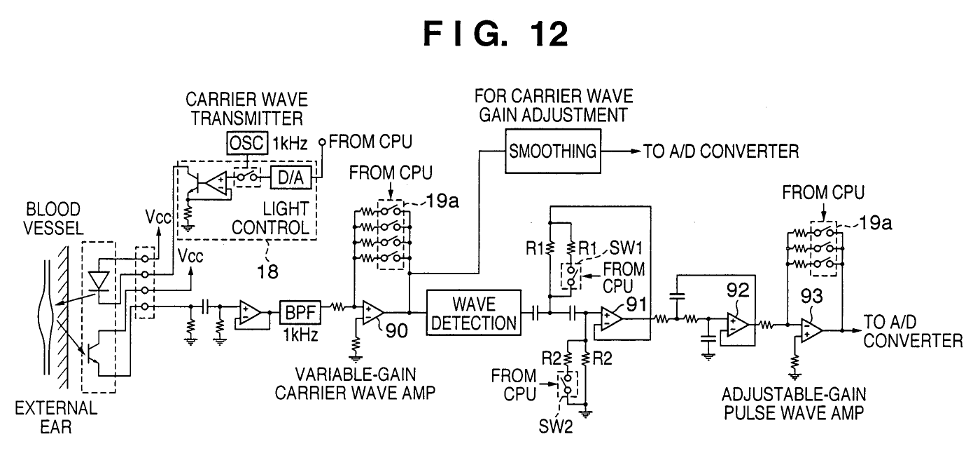

FIG. 10 is an operation flowchart of signal level adjustment in the blood pressure measuring apparatus according to the fourth embodiment. FIG. 12 is an exemplary circuit diagram related to the signal level adjustment.

When adjusting the light quantity and gain, in Step S1001, the apparatus closes (turns on) SW1 and SW2 in FIG. 12, thereby reducing resistance to half, and thereby halves the time constant of filter amplifiers 91 and 92. In this state, the apparatus detects a carrier wave level in Step S1002 and checks in Step S1003 whether a carrier wave of the pulse wave is within a specified range (20 to 40% the full scale of A/D 10). If the carrier wave is below the specified range, the apparatus goes to Step S1004 to check whether the light quantity is a maximum. If it is not a maximum, the apparatus makes the light controller 18 increase the light quantity in Step S1006. If the light quantity is a maximum, the apparatus increases the gain by controlling feedback of an amplifier 90 in Step S1005. After the process in Step S1005 or Step S1006, the apparatus returns to Step S1102 to check the carrier wave level again.

On the other hand, if it is found in Step S1003 that the carrier wave level is above the specified range, the apparatus checks in Step S1007 whether or not the gain is a minimum. If is it not a minimum, the apparatus makes the gain controller 19a decrease the gain by controlling the feedback of the amplifier 90 in Step S1009. If the gain is a minimum, the apparatus decreases the light quantity in Step S1008. When the process in Step S1008 or Step S1009 is finished, the apparatus returns to Step S1002 to check the carrier wave level again. If it is found in Step S1003 that the carrier wave level is within the specified range, the apparatus opens SW1 and SW2 in Step S1010 to restore the time constant of filter amplifiers 91 and 92 and adjusts the gain of the pulse wave using an amplifier 93 in Step S1011.

Although an example of detecting light reflected by the blood in blood vessels has been shown in this embodiment, transmitted light may be detected alternatively.

As described above, this embodiment provides a photoelectric sphygmomanometer which makes it possible to adjust the signal level of a pulse wave signal so that the signal level will fall within a specified range, thereby enabling high-accuracy measurements, and reduce the time of blood pressure measurements, thereby reducing the physical burden imposed on the user by cuff pressure. Since a tragus and its surroundings are impervious to pain, this embodiment has the advantage of reducing pain cause by cuff pressure. Consequently, this embodiment can be applied easily to continuous measurement of blood pressure.

### Fifth Embodiment

A fifth embodiment takes measurements only during pressurization and provides high-accuracy blood pressure measurements by adjusting the light quantity and gain based on a pulse wave signal obtained before the blood pressure measurements during the pressurization.

Incidentally, equipment configuration, a method of attaching the cuff to a measurement site, calculation of blood pressure, and details of adjusting the light quantity and gain of the apparatus are the same as in the fourth embodiment, and a description thereof will be omitted.

<Equipment Operation>

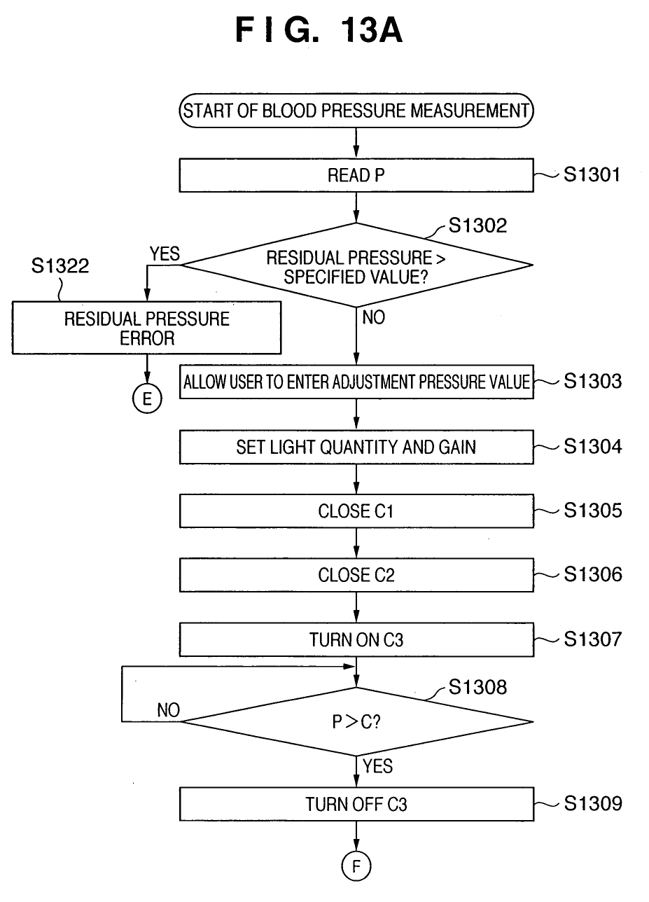

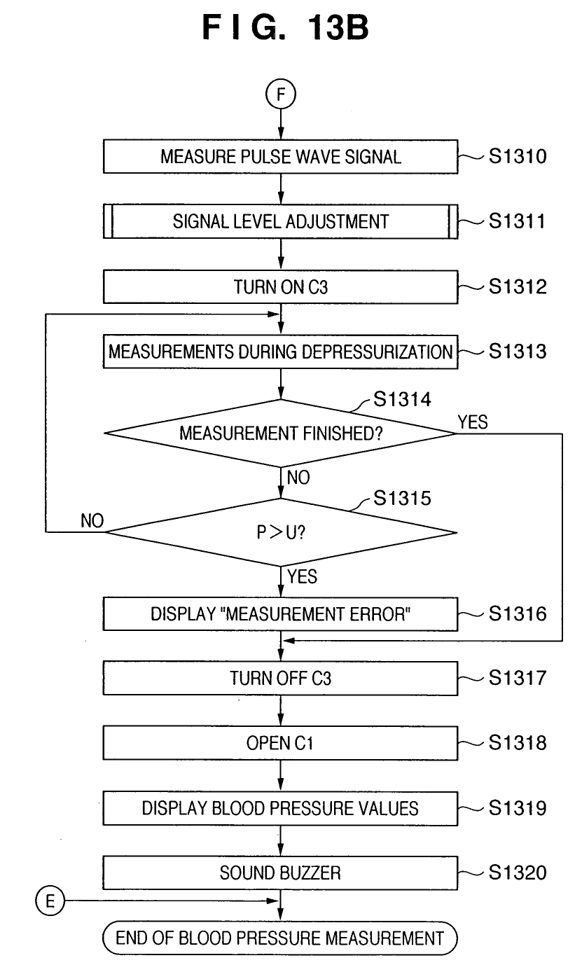

FIGS. 13A and 13B are operation flowcharts of the blood pressure measuring apparatus according to the fifth embodiment. When the apparatus is turned on by the power switch 30, it initializes itself by performing a self-diagnosis process (not shown). Subsequently, when the measurement start switch ST is pressed, the apparatus starts processing.

In Step S1301, the apparatus reads the cuff pressure P. In Step S1302, the apparatus compares residual pressure of the cuff 1 with a specified value. If the residual pressure exceeds the specified value, the apparatus displays “residual pressure error” on the LCD 14 in Step S1322. If the residual pressure is not higher than the specified value, the apparatus allows the user in Step S1303 to set a pressurization value (e.g., a value between 120 and 210 mmHg, which is higher than a systolic blood pressure) of the cuff using the keyboard 16. In Step S1304, the apparatus sets the light quantity and gain to predetermined values.

When the light quantity and gain have been set, the apparatus closes the quick exhaust valve 4 and slow exhaust valve 5 in Steps S1305 and S1306, respectively. In Step S1307, the apparatus starts operating the pressure pump 3, and thereby starts pressurization (compression).

In Step S1308, the apparatus determines whether or not the cuff pressure is higher than the pressurization value C set in Step S1303. If P>C is not met, the apparatus continues pressurization. If P>C, the apparatus stops the pressure pump 3 in Step S1309. The apparatus obtains a pulse wave signal using the sensor 8 in Step S1310 and sets the light quantity and gain again in Step S1311 to such values which will give a predetermined signal level. In Step S1312, the apparatus starts operating the pressure pump 3, and thereby resumes pressurization. This marks the start of a measurement stroke during pressurization. The cuff pressure starts to increase at a constant rate (e.g., 2 to 3 mmHg/sec). Meanwhile, in Step S1313, various functional blocks perform data processing, and the apparatus measures systolic and diastolic blood pressures. Once the systolic blood pressure has been measured (Step S1314), the apparatus stops the pressure pump 3 in Step S1317 and discharges the remaining air rapidly from the cuff 1 in Step S1318.

In Step S1315, the apparatus determines whether or not the cuff pressure is higher than the pressurization value U set in Step S1303. If P>U is not met, the cuff pressure is still within a normal measuring range and thus the apparatus continues measurement. On the other hand, if P>U, the cuff pressure is already higher than the set value. Thus, the apparatus displays “measurement error” on the LCD 14 in Step S1316. The apparatus additionally displays detailed information such as “signal failure during pressurization” if necessary.

If it is found in Step S1314 that measurements have been finished, meaning that the measurement stroke has been finished within a normal measuring range, the apparatus displays the measured systolic and diastolic blood pressures on the LCD 14 in Step S1319 and sends a tone signal to the buzzer 15 in Step S1320. Preferably, different tone signals are sent for a normal end and an abnormal end.

As described above, the photoelectric sphygmomanometer according to this embodiment makes it possible to adjust the signal level of a pulse wave signal so that the signal level will fall within a specified range, thereby enabling proper blood pressure measurements. Also, it has the advantage of reducing the need to measure blood pressure again during depressurization. Besides, by further reducing the time of blood pressure measurements, this embodiment reduces the physical burden imposed on the user by cuff pressure.

### Sixth Embodiment

In a sixth embodiment, description will be given of a blood pressure measuring apparatus which can measure multiple sites at a time.

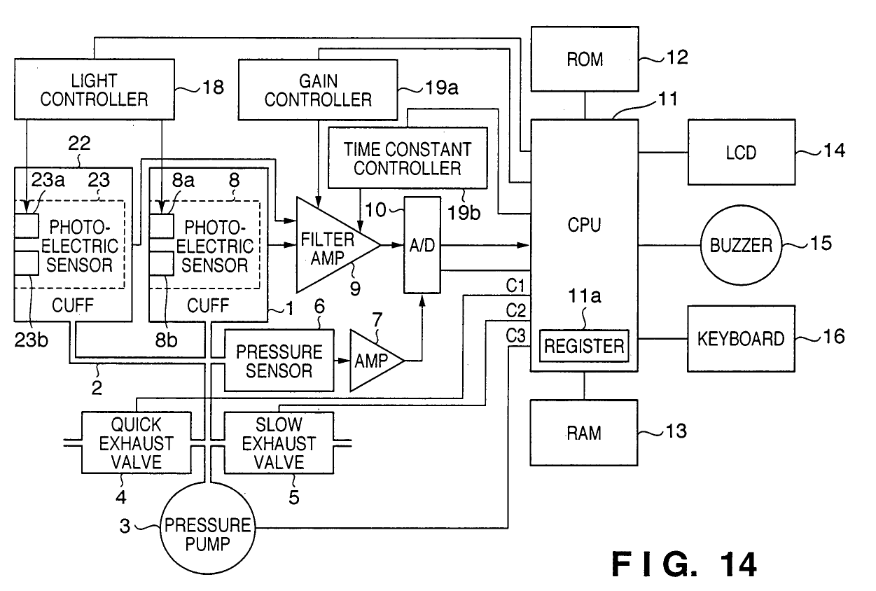

FIG. 14 is an internal block diagram of a blood pressure measuring apparatus according to a sixth embodiment. Each cuff which pinches a tragus and/or its surroundings is equipped with a light-emitting unit (see FIG. 14: LED 8a or 21a) and light-receiving unit (see FIG. 14: phototransistor 8b or 21b). The two cuffs are configured to be pressurized by a single pressure pump 3 to measure blood pressure at multiple sites on and/or around a tragus, i.e., at two measurement sites on the front (inner) and back (outer) sides of the tragus, simultaneously.

As shown in FIG. 14, the blood pressure measuring apparatus according to the sixth embodiment has a pulse wave sensor 23 in another cuff 22 in addition to the equipment configuration of the fourth embodiment (FIG. 8). The cuff 22 contains a LED 23a which illuminates pulsating vascular blood flow with light and phototransistor 23b which detects light reflected by the vascular blood flow. Incidentally, sensors based on different measurement principles (the pressure plethysmographic method and the like) may be used for blood pressure measurements. The rest of the configuration and operation is the same as in the first and second embodiments, and a description thereof will be omitted.

It is known that blood vessels (arterioles) in and/or around the tragi are located in close vicinity to blood vessels in the brain, and it is considered that changes in blood pressure resulting from intracerebral causes can be measured. On the other hand, around the tragi, there are not only blood vessels (arterioles) in the ear cartilage (mainly tragi), but also arteries (superficial temporal artery) directly connected to the heart. This offers the advantage of being able to measure blood pressure carrying different pieces of information (blood pressure attributable to the brain and blood pressure attributable to the heart) simultaneously around a tragus using a small apparatus. The photoelectric sphygmomanometer according to this embodiment makes it possible to adjust the signal level of a pulse wave signal so that the signal level will fall within a specified range, thereby enabling high-accuracy blood pressure measurements around an external ear. At the same time, by further reducing the time of blood pressure measurements, this embodiment reduces the physical burden imposed on the user by cuff pressure.

The present invention is not limited to the above embodiments and various changes and modifications can be mad within the spirit and scope of the present invention. Therefore, to apprise the public of the scope of the present invention, the following claims are made.

### CLAIM OF PRIORITY

This application claims priority from Japanese patent applications 2004-294307 and 2004-294308 filed Oct. 6, 20004, the disclosure of which is herein incorporated by reference in its entirety.

1. A blood pressure measuring apparatus comprising:
a cuff which is attached to and around an external ear;
a first pulse wave detector and a second pulse wave detector which detect a pulse wave in a part squeezed by said cuff and which are affected differently from each other by a characteristic of body movements;
body movement detecting means which detects the characteristic of body movements;
pulse wave selecting means which selects a pulse wave detected by one of said first pulse wave detector and said second pulse wave detector based on the characteristic of body movements detected by said body movement detecting means; and
blood pressure value deriving means which derives a blood pressure value based on the pulse wave selected by said pulse wave selecting means.

2. The blood pressure measuring apparatus according to claim 1, wherein:
said body movement detecting means comprises level detecting means which detects magnitude of the body movements; and
said pulse wave selecting means selects a pulse wave for use to derive blood pressure based on the magnitude of body movements detected by said level detecting means.

3. The blood pressure measuring apparatus according to claim 2, wherein:
said body movement detecting means further comprises a period detecting means which detects a period of the body movements; and
said pulse wave selecting means selects a pulse wave for use to derive blood pressure based on the magnitude of body movements detected by said level detecting means and the period of the body movements detected by said period detecting means.

4. A blood pressure measuring apparatus comprising:
a first cuff which is attached to and around an external ear;
a first pulse wave detector and a second pulse wave detector which detect a pulse wave in a part squeezed by said first cuff and which are affected differently from each other by a characteristic of body movements;
body movement detecting means which detects the characteristic of body movements;
first pulse wave selecting means which selects a pulse wave detected by one of said first pulse wave detector and said second pulse wave detector based on the characteristic of body movements detected by said body movement detecting means;
first blood pressure value deriving means which derives a blood pressure value based on the pulse wave selected by said first pulse wave selecting means;
a second cuff mounted in a different location from said first cuff; and
blood pressure determining means which determines blood pressure by detecting a pulse wave in a part squeezed by said second cuff; and
pressurization control means which synchronizes pressurization of said first cuff and said second cuff.

5. The blood pressure measuring apparatus according to claim 4, wherein said blood pressure determining means comprises:
a third pulse wave detector and a fourth pulse wave detector which detect a pulse wave in the part squeezed by said second cuff and which are affected differently from each other by a characteristic of body movements;
second pulse wave selecting means which selects a pulse wave detected by one of said third pulse wave detector and said fourth pulse wave detector based on the characteristic of body movements detected by said body movement detecting means; and
second blood pressure value deriving means which derives a blood pressure value based on the pulse wave selected by said second pulse wave selecting means.

6. A blood pressure measuring apparatus comprising:
a cuff which is attached to and around an external ear;
a pulse wave detector which detects a pulse wave in a part squeezed by said cuff;
level control means which controls a signal level of the pulse wave; and
blood pressure derivation control means which makes adjustments using said level control means so as to correct any deviation of the signal level of the pulse wave detected by said pulse wave detector during compression of said cuff from a predetermined range and derives a blood pressure value based on the pulse wave detected by said pulse wave detector during decompression of said cuff.

7. The blood pressure measuring apparatus according to claim 6, wherein if the signal level of the pulse wave detected by said pulse wave detector during compression of said cuff falls within the predetermined range, said blood pressure derivation control means derives a blood pressure value based on the pulse wave and finishes a measurement operation.

8. The blood pressure measuring apparatus according to claim 6, wherein the pulse wave detected by said pulse wave detector is a photoelectric volume pulse wave obtained via light absorption and reflection by blood in blood vessels.

9. The blood pressure measuring apparatus according to claim 8, wherein said level control means comprises at least one of light quantity adjusting means which adjusts quantity of output light from a light-emitting element which emits light to the blood in the blood vessels and gain control means which controls a signal level from a light-receiving element which detects absorption and reflection of the light from the light-emitting element by the blood in the blood vessels.

10. A blood pressure measuring apparatus comprising:
a cuff which is attached to and around an external ear;
a pulse wave detector which detects a pulse wave in a part squeezed by said cuff;
level control means which controls a signal level of the pulse wave; and
blood pressure derivation control means which makes adjustments using said level control means so as to correct any deviation of the signal level of the pulse wave detected by said pulse wave detector before compression or at an early stage of compression of said cuff from a predetermined range and derives a blood pressure value based on the pulse wave detected by said pulse wave detector during subsequent compression of said cuff.

11. The blood pressure measuring apparatus according to claim 10, wherein the pulse wave detected by said pulse wave detector is a photoelectric volume pulse wave obtained via light absorption and reflection by blood in blood vessels.

12. The blood pressure measuring apparatus according to claim 11, wherein said level control means comprises at least one of light quantity adjusting means which adjusts quantity of output light from a light-emitting element which emits light to the blood in the blood vessels and gain control means which controls a signal level from a light-receiving element which detects absorption and reflection of the light from the light-emitting element by the blood in the blood vessels.

13. A blood pressure measuring apparatus comprising:
a first cuff which is attached to and around an external ear;
a pulse wave detector which detects a pulse wave in a part squeezed by said first cuff;
level control means which controls a signal level of the pulse wave;
blood pressure derivation control means which makes adjustments using said level control means so as to correct any deviation of the signal level of the pulse wave detected by said pulse wave detector during compression of said first cuff from a predetermined range and derives a blood pressure value based on the pulse wave detected by said pulse wave detector during decompression of said first cuff;
a second cuff mounted in a different location from said first cuff;
blood pressure determining means which determines blood pressure by detecting a pulse wave in a part squeezed by said second cuff; and
pressurization control means which synchronizes pressurization of said first cuff and said second cuff.

14. A blood pressure measuring apparatus comprising:
a first cuff which is attached to and around an external ear;
a pulse wave detector which detects a pulse wave in a part squeezed by said first cuff;
level control means which controls a signal level of the pulse wave;
blood pressure derivation control means which makes adjustments using said level control means so as to correct any deviation of the signal level of the pulse wave detected by said pulse wave detector before compression or at an early stage of compression of said first cuff from a predetermined range and derives a blood pressure value based on the pulse wave detected by said pulse wave detector during subsequent compression of said first cuff;
a second cuff mounted in a different location from said first cuff;
blood pressure determining means which determines blood pressure by detecting a pulse wave in a part squeezed by said second cuff; and
pressurization control means which synchronizes pressurization of said first cuff and said second cuff.

15. A blood pressure measuring method comprising:
a pulse wave detecting step of detecting a first pulse wave and a second pulse wave in a part squeezed by a cuff attached to and around an external ear, the first pulse wave and the second pulse wave being affected differently from each other by a characteristic of body movements;
a body movement detecting step of detecting the characteristic of body movements;
a pulse wave selecting step of selecting one of the first pulse wave and the second pulse wave based on the characteristic of body movements detected by said body movement detecting step; and
a blood pressure value deriving step of deriving a blood pressure value based on the pulse wave selected by said pulse wave selecting step.

16. The blood pressure measuring method according to claim 15, wherein:
said body movement detecting step comprises a level detecting step of detecting magnitude of the body movements; and
said pulse wave selecting step selects a pulse wave for use to derive blood pressure based on the magnitude of body movements detected by said level detecting step.

17. The blood pressure measuring method according to claim 16, wherein:
said body movement detecting step further comprises a period detecting step of detecting a period of the body movements; and
said pulse wave selecting step selects a pulse wave for use to derive blood pressure based on the magnitude of body movements detected by said level detecting step and the period of the body movements detected by said period detecting step.

18. A blood pressure measuring method comprising:
a compression-time pulse wave detecting step of detecting a pulse wave in a part squeezed by a cuff attached to and around an external ear during compression of the cuff;
a level control step of controlling a signal level of the pulse wave so as to correct any deviation of the signal level of the pulse wave detected by said compression-time pulse wave detecting step from a predetermined range;
a decompression-time pulse wave detecting step of detecting a pulse wave in a part squeezed by the cuff during decompression of the cuff; and
a blood pressure value deriving step of deriving a blood pressure value based on a pulse wave whose signal level, as detected by said compression-time pulse wave detecting step or said decompression-time pulse wave detecting step, falls within a predetermined range.

19. The blood pressure measuring method according to claim 18, wherein if the signal level of the pulse wave detected by said compression-time pulse wave detecting step falls within a predetermined range, said blood pressure value deriving step derives a blood pressure value based on the pulse wave detected by said compression-time pulse wave detecting step without regard to said level control step and said decompression-time pulse wave detecting step.

20. The blood pressure measuring method according to claim 18, wherein the pulse waves detected by said compression-time pulse wave detecting step and said decompression-time pulse wave detecting step are photoelectric volume pulse waves obtained via light absorption and reflection by blood in blood vessels.

21. The blood pressure measuring method according to claim 20, wherein said level control step comprises at least one of a light quantity adjusting step of adjusting quantity of output light from a light-emitting element which emits light to the blood in the blood vessels and a gain control step of controlling a signal level from a light-receiving element which detects absorption and reflection of the light from the light-emitting element by the blood in the blood vessels.

22. A blood pressure measuring method comprising:
an initial pulse wave detecting step of detecting a pulse wave in a part squeezed by a cuff attached to and around an external ear before compression or at an early stage of compression of said cuff;
a level control step of controlling a signal level of the pulse wave so as to correct any deviation of the signal level of the pulse wave detected by said initial pulse wave detecting step from a predetermined range;
a pulse wave detecting step of detecting a pulse wave in the part squeezed by said cuff during subsequent compression of said cuff; and
a blood pressure value deriving step of deriving a blood pressure value based on the pulse wave detected by said pulse wave detecting step.

23. The blood pressure measuring method according to claim 22, wherein the pulse wave detected by said pulse wave detecting step is a photoelectric volume pulse wave obtained via light absorption and reflection by blood in blood vessels.

24. The blood pressure measuring method according to claim 23, wherein said level control step comprises at least one of a light quantity adjusting step of adjusting quantity of output light from a light-emitting element which emits light to the blood in the blood vessels and a gain control step of controlling a signal level from a light-receiving element which detects absorption and reflection of the light from the light-emitting element by the blood in the blood vessels.
